(19) United States
(12) Patent Application Publication
(10) Pub. No.: US 2006/0253130 A1
(43) Pub. Date: Nov. 9, 2006

(54) Tissue fixation assemblies having a plurality of fasteners ready for serial deployment

(75) Inventor: Raymond Michael Wolniewicz III, Redmond, WA (US)

Correspondence Address: GRAYBEAL JACKSON HALEY LLP, Suite. 350, 155-108th Avenue N.E., Bellevue, WA, 98004-5973, US

(73) Assignee: EsophyX, Inc.

(21) Appl. No.: 11/121,697

(22) Filed: May 3, 2005

(51) Int. Cl. A61B 17/10

(52) U.S. Cl. 606/142

(57) ABSTRACT

A plurality of tissue fasteners are carried on a tissue piercing stylet to serially fasten tissue layers of a mammalian body together. The fasteners slide over a latch of the stylet to assume a loaded position ready for deployment. The fasteners may be deployed with a single stroke movement of the stylet. The fasteners have a configuration alterable by the stylet to permit release of the fasteners from the stylet after deployment.

### CROSS-REFERENCE TO RELATED APPLICATION

This application is related to U.S. patent application Ser. No. ______ (Atty. Docket No. 2234-12-3) entitled “TISSUE FIXATION ASSEMBLIES PROVIDING SINGLE STROKE DEPLOYMENT”, which was filed on the same day as the present application and which is incorporated by reference.

### FIELD OF THE INVENTION

The present invention generally relates to tissue fixation devices and fastener assemblies which may be used, for example, for treating gastroesophageal reflux disease. The present invention more particularly relates to such tissue fixation assemblies which may deploy a fastener for fixing tissue with but a single translational movement of a fastener and a stylet.

### BACKGROUND

Gastroesophageal reflux disease (GERD) is a chronic condition caused by the failure of the anti-reflux barrier located at the gastroesophageal junction to keep the contents of the stomach from splashing into the esophagus. The splashing is known as gastroesophageal reflux. The stomach acid is designed to digest meat, and will digest esophageal tissue when persistently splashed into the esophagus.

A principal reason for regurgitation associated with GERD is the mechanical failure of a deteriorated gastroesophageal flap to close and seal against high pressure in the stomach. Due to reasons including lifestyle, a Grade I normal gastroesophageal flap may deteriorate into a malfunctioning Grade III or absent valve Grade IV gastroesophageal flap. With a deteriorated gastroesophageal flap, the stomach contents are more likely to be regurgitated into the esophagus, the mouth, and even the lungs. The regurgitation is referred to as “heartburn” because the most common symptom is a burning discomfort in the chest under the breastbone. Burning discomfort in the chest and regurgitation (burping up) of sour-tasting gastric juice into the mouth are classic symptoms of gastroesophageal reflux disease (GERD). When stomach acid is regurgitated into the esophagus, it is usually cleared quickly by esophageal contractions. Heartburn (backwashing of stomach acid and bile onto the esophagus) results when stomach acid is frequently regurgitated into the esophagus and the esophageal wall is inflamed.

Complications develop for some people who have GERD. Esophagitis (inflammation of the esophagus) with erosions and ulcerations (breaks in the lining of the esophagus) can occur from repeated and prolonged acid exposure. If these breaks are deep, bleeding or scarring of the esophagus with formation of a stricture (narrowing of the esophagus) can occur. If the esophagus narrows significantly, then food sticks in the esophagus and the symptom is known as dysphagia. GERD has been shown to be one of the most important risk factors for the development of esophageal adenocarcinoma. In a subset of people who have severe GERD, if acid exposure continues, the injured squamous lining is replaced by a precancerous lining (called Barrett's Esophagus) in which a cancerous esophageal adenocarcinoma can develop.

Other complications of GERD may not appear to be related to esophageal disease at all. Some people with GERD may develop recurrent pneumonia (lung infection), asthma (wheezing), or a chronic cough from acid backing up into the esophagus and all the way up through the upper esophageal sphincter into the lungs. In many instances, this occurs at night, while the person is in a supine position and sleeping. Occasionally, a person with severe GERD will be awakened from sleep with a choking sensation. Hoarseness can also occur due to acid reaching the vocal cords, causing a chronic inflammation or injury.

GERD never improves without intervention. Life style changes combined with both medical and surgical treatments exist for GERD. Medical therapies include antacids and proton pump inhibitors. However, the medical therapies only mask the reflux. Patients still get reflux and perhaps emphysema because of particles refluxed into the lungs. Barrett's esophagus results in about 10% of the GERD cases. The esophageal epithelium changes into tissue that tends to become cancerous from repeated acid washing despite the medication.

Several open laparotomy and laproscopic surgical procedures are available for treating GERD. One surgical approach is the Nissen fundoplication. The Nissen approach typically involves a 360-degree wrap of the fundus around the gastroesophageal junction. The procedure has a high incidence of postoperative complications. The Nissen approach creates a 360-degree moveable flap without a fixed portion. Hence, Nissen does not restore the normal movable flap. The patient cannot burp because the fundus was used to make the repair, and may frequently experience dysphagia. Another surgical approach to treating GERD is the Belsey Mark IV (Belsey) fundoplication. The Belsey procedure involves creating a valve by suturing a portion of the stomach to an anterior surface of the esophagus. It reduces some of the postoperative complications encountered with the Nissen fundoplication, but still does not restore the normal movable flap. None of these procedures fully restores the normal anatomical anatomy or produces a normally functioning gastroesophageal junction. Another surgical approach is the Hill repair. In the Hill repair, the gastroesophageal junction is anchored to the posterior abdominal areas, and a 180-degree valve is created by a system of sutures. The Hill procedure restores the moveable flap, the cardiac notch and the Angle of His. However, all of these surgical procedures are very invasive, regardless of whether done as a laproscopic or an open procedure.

New, less surgically invasive approaches to treating GERD involve transoral endoscopic procedures. One procedure contemplates a machine device with robotic arms that is inserted transorally into the stomach. While observing through an endoscope, an endoscopist guides the machine within the stomach to engage a portion of the fundus with a corkscrew-like device on one arm. The arm then pulls on the engaged portion to create a fold of tissue or radial plication at the gastroesophageal junction. Another arm of the machine pinches the excess tissue together and fastens the excess tissue with one pre-tied implant. This procedure does not restore normal anatomy. The fold created does not have anything in common with a valve. In fact, the direction of the radial fold prevents the fold or plication from acting as a flap of a valve.

Another transoral procedure contemplates making a fold of fundus tissue near the deteriorated gastroesophageal flap to recreate the lower esophageal sphincter (LES). The procedure requires placing multiple U-shaped tissue clips around the folded fundus to hold it in shape and in place.

This and the previously discussed procedure are both highly dependent on the skill, experience, aggressiveness, and courage of the endoscopist. In addition, these and other procedures may involve esophageal tissue in the repair. Esophageal tissue is fragile and weak, in part due to the fact, that the esophagus is not covered by serosa, a layer of very sturdy, yet very thin tissue, covering and stabilizing all intraabdominal organs, similar like a fascia covering and stabilizing muscle. Involvement of esophageal tissue in the repair of a gastroesophageal flap valve poses unnecessary risks to the patient, such as an increased risk of fistulas between the esophagus and the stomach.

A new and improved apparatus and method for restoration of a gastroesophageal flap valve is fully disclosed in U.S. Pat. No. 6,790,214, issued Sep. 14, 2004, is assigned to the assignee of this invention, and is incorporated herein by reference. That apparatus and method provides a transoral endoscopic gastroesophageal flap valve restoration. A longitudinal member arranged for transoral placement into a stomach carries a tissue shaper that non-invasively grips and shapes stomach tissue. A tissue fixation device is then deployed to maintain the shaped stomach tissue in a shape approximating a gastroesophageal flap.

Whenever tissue is to be maintained in a shape as, for example, with the improved assembly last mentioned above, it is necessary to fasten at least two layers of tissue together. In applications such as gastroesophageal flap valve restoration, there is very limited room to maneuver a fastener deployment device. For example, this and other medical fastening applications provide confined working channels and spaces and often must be fed through an endoscope to permit visualization or other small lumen guide catheters to the place where the fasteners are to be deployed. To make matters worse, multiple fasteners may also be required. Hence, with current fasteners and deployment arrangements, it is often difficult to direct a single fastener to its intended location, let alone a number of such fasteners.

Once the fastening site is located, the fasteners employed must be truly able to securely maintain the tissue. Still further, the fastener must be readily deployable. Also, quite obviously, the fasteners are preferably deployable in the tissue in a manner which does not unduly traumatize the tissue.

Improved fasteners and systems for deploying the same are fully disclosed in copending application Ser. No. 11/043,903, filed Jan. 25, 2005, for SLITTED TISSUE FIXATION DEVICES AND ASSEMBLIES FOR DEPLOYING THE SAME, which application is incorporated herein by reference. The assembly includes a fastener including a first member, and a second member. The first and second members have first and second ends. The fastener further comprises a connecting member fixed to each of the first and second members intermediate the first and second ends and extends between and separates the first and second members. The first member has a longitudinal axis, a through channel along the axis, and a slit extending between the first and second ends and communicating with the through channel. A deployment wire or stylet is arranged to be slidingly received by the through channel of the first member and has a pointed tip to pierce into tissue. The stylet thus guides the fastener to the fastening location when a pusher pushes the first member into the tissue while on the deployment wire. As the first member is driven into the tissue by the pusher, the second member engages to tissue. This provides resistance against further movement of the fastener. Continued pushing of the fastener causes the first member to be deformed by the stylet. As the first member pivots on the connecting member, the stylet is forced out of the first member either by passing through the first member slit, the deformation of the first member, or a combination of these factors.

As can thus be appreciated, deployment of the fastener requires manipulation of both the pusher and stylet simultaneously. It would be desirable if the fastener deployment could be made easier by negating the need for the simultaneous manipulations. The present invention addresses this and other issues as will be seen subsequently.

### SUMMARY

The invention provides a fastener assembly for use in a mammalian body. The fastener comprises a plurality of fasteners, each fastener being arranged to fasten tissue when deployed, and a stylet that guides each fastener into tissue to be fastened. The plurality of fasteners are carried on the stylet prior to deployment.

The fasteners may include a through channel that slidingly receives the stylet. Each of the fasteners may further include a first member, a second member, and a connecting member connecting the first member to the second member. The first and second members may each have first and second ends. The connecting member may connect to the first and second members intermediate the first and second ends of each of the first and second members.

The assembly may further comprise a drive structure that drives the fasteners into the tissue while the fasteners are carried on the stylet. The stylet may include a pointed tip that pierces the tissue before the fasteners pierce the tissue.

According to one embodiment, the fasteners may include a driven member that is driven into the tissue while on the stylet during deployment, a second member, and a connecting member connecting the driven member and second member. When a fastener is deployed, the tissue is between the driven member and the second member and the connecting member extends through the tissue.

The driven member of each fastener is preferably releasable from the stylet. The fastener may include a through channel that slidingly receives the stylet and that permits the fasteners to be carried on the stylet. The fasteners may further include a slit communicating with their through channel. The fasteners may then be releasable from the stylet by the stylet passing through their slit.

The assembly may further comprise a guide defining a guide lumen. The guide lumen is adapted to receive the fasteners and stylet and guide the stylet and fasteners to the tissue.

The invention further provides a fastener assembly for use in a mammalian body comprising a plurality of fasteners. Each fastener is arranged to fasten tissue when deployed and includes a driven member, a second member, and a connecting member connecting the driven member to the second member. The driven and second members each have first and second ends with the connecting member connecting to the driven and second members intermediate the first and second ends of each of the driven and second members. The driven member may also include a through channel. A stylet guides each fastener into tissue to be fastened. The plurality of fasteners are carried on the stylet with the through channel of the driven member of each fastener slidingly received on the stylet.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like reference numerals identify like elements, and wherein:

FIG. 1 is a front cross-sectional view of the esophageal-gastro-intestinal tract from a lower portion of the esophagus to the duodenum;

FIG. 2 is a front cross-sectional view of the esophageal-gastro-intestinal tract illustrating a Grade I normal appearance movable flap of the gastroesophageal flap valve (in dashed lines) and a Grade III reflux appearance gastroesophageal flap of the gastroesophageal flap valve (in solid lines);

FIG. 3 is a perspective view of a fastener embodying the invention;

FIG. 4 is a perspective view with portions cut away of a fastener assembly according to an embodiment of the invention in an early stage of deploying the fastener of FIG. 3;

FIG. 5 is a perspective view of the assembly of FIG. 4 shown with the fastener being driven in the tissue layers to be fastened;

FIG. 6 is a perspective view of the assembly of FIG. 4 shown with the fastener in an intermediate stage of deployment;

FIG. 7 is a perspective view of the assembly of FIG. 4 shown with the fastener almost completely deployed;

FIG. 8 is a perspective view showing the fastener of the assembly of FIG. 4 fully deployed and securely fastening a pair of tissue layers together;

FIG. 9 is a side view of a fastener according to a further embodiment of the present invention;

FIG. 10 is a side view of another fastener according to another embodiment of the present invention;

FIG. 11 is a perspective view with portions cut away of a fastener assembly according to another embodiment of the invention;

FIG. 12 is a perspective view of the assembly of FIG. 11 after having deployed a fastener; and

FIG. 13 is a partial perspective view of a stylet having an integral spring loaded latch according to another embodiment of the present invention.

### DETAILED DESCRIPTION

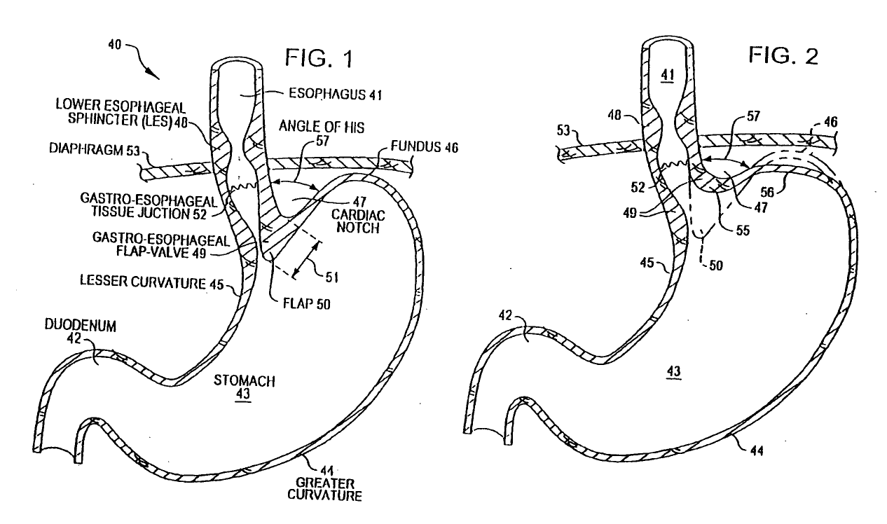

FIG. 1 is a front cross-sectional view of the esophageal-gastro-intestinal tract 40 from a lower portion of the esophagus 41 to the duodenum 42. The stomach 43 is characterized by the greater curvature 44 on the anatomical left side and the lesser curvature 45 on the anatomical right side. The tissue of the outer surfaces of those curvatures is referred to in the art as serosa tissue. As will be seen subsequently, the nature of the serosa tissue is used to advantage for its ability to bond to like serosa tissue. The fundus 46 of the greater curvature 44 forms the superior portion of the stomach 43, and traps gas and air bubbles for burping. The esophageal tract 41 enters the stomach 43 at an esophageal orifice below the superior portion of the fundus 46, forming a cardiac notch 47 and an acute angle with respect to the fundus 46 known as the Angle of His 57. The lower esophageal sphincter (LES) 48 is a discriminating sphincter able to distinguish between burping gas, liquids, and solids, and works in conjunction with the fundus 46 to burp. The gastroesophageal flap valve (GEFV) 49 includes a moveable portion and an opposing more stationary portion. The moveable portion of the GEFV 49 is an approximately 180 degree, semicircular, gastroesophageal flap 50 (alternatively referred to as a “normal moveable flap” or “moveable flap”) formed of tissue at the intersection between the esophagus 41 and the stomach 43. The opposing more stationary portion of the GEFV 49 comprises a portion of the lesser curvature 45 of the stomach 43 adjacent to its junction with the esophagus 41. The gastroesophageal flap 50 of the GEFV 49 principally comprises tissue adjacent to the fundus 46 portion of the stomach 43, is about 4 to 5 cm long (51) at it longest portion, and the length may taper at its anterior and posterior ends. The gastroesophageal flap 50 is partially held against the lesser curvature 45 portion of the stomach 43 by the pressure differential between the stomach 43 and the thorax, and partially by the resiliency and the anatomical structure of the GEFV 49, thus providing the valving function. The GEFV 49 is similar to a flutter valve, with the gastroesophageal flap 50 being flexible and closeable against the other more stationary side.

The esophageal tract is controlled by an upper esophageal sphincter (UES)in the neck near the mouth for swallowing, and by the LES 48 and the GEFV 49 at the stomach. The normal anti-reflux barrier is primarily formed by the LES 48 and the GEFV 49 acting in concert to allow food and liquid to enter the stomach, and to considerably resist reflux of stomach contents into the esophagus 41 past the gastroesophageal tissue junction 52. Tissue aboral of the gastroesophageal tissue junction 52 is generally considered part of the stomach because the tissue protected from stomach acid by its own protective mechanisms. Tissue oral of the gastroesophageal junction 52 is generally considered part of the esophagus and it is not protected from injury by prolonged exposure to stomach acid. At the gastroesophageal junction 52, the juncture of the stomach and esophageal tissues form a zigzag line, which is sometimes referred to as the “Z-line.” For the purposes of these specifications, including the claims, “stomach” means the tissue aboral of the gastroesophageal junction 52.

FIG. 2 is a front cross-sectional view of an esophageal-gastro-intestinal tract illustrating a Grade I normal appearance movable flap 50 of the GEFV 49 (shown in dashed lines) and a deteriorated Grade III gastroesophageal flap 55 of the GEFV 49 (shown in solid lines). As previously mentioned, a principal reason for regurgitation associated with GERD is the mechanical failure of the deteriorated (or reflux appearance) gastroesophageal flap 55 of the GEFV 49 to close and seal against the higher pressure in the stomach. Due to reasons including lifestyle, a Grade I normal gastroesophageal flap 50 of the GEFV 49 may deteriorate into a Grade III deteriorated gastroesophageal flap 55. The anatomical results of the deterioration include moving a portion of the esophagus 41 that includes the gastroesophageal junction 52 and LES 48 toward the mouth, straightening of the cardiac notch 47, and increasing the Angle of His 57. This effectively reshapes the anatomy aboral of the gastroesophageal junction 52 and forms a flattened fundus 56. The deteriorated gastroesophageal flap 55 illustrates a gastroesophageal flap valve 49 and cardiac notch 47 that have both significantly degraded. Dr. Hill and colleagues developed a grading system to describe the appearance of the GEFV and the likelihood that a patient will experience chronic acid reflux. L. D. Hill, et al., The gastroesophageal flap valve: in vitro and in vivo observations, Gastrointestinal Endoscopy 1996:44:541-547. Under Dr. Hill's grading system, the normal movable flap 50 of the GEFV 49 illustrates a Grade I flap valve that is the least likely to experience reflux. The deteriorated gastroesophageal flap 55 of the GEFV 49 illustrates a Grade III (almost Grade IV) flap valve. A Grade IV flap valve is the most likely to experience reflux. Grades II and III reflect intermediate grades of deterioration and, as in the case of III, a high likelihood of experiencing reflux. With the deteriorated GEFV represented by deteriorated gastroesophageal flap 55 and the fundus 46 moved inferior, the stomach contents are presented a funnel-like opening directing the contents into the esophagus 41 and the greatest likelihood of experiencing reflux. Disclosed subsequently is a fastener and assembly which may be employed to advantage in restoring the normal gastroesophageal flap valve anatomy.

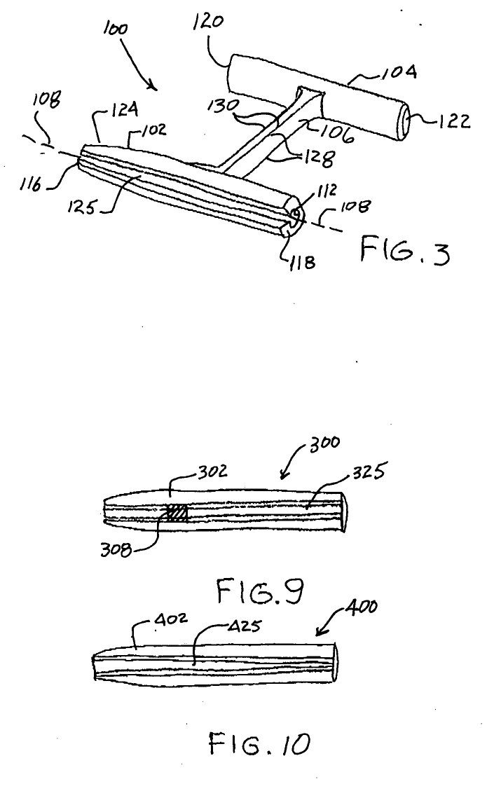

FIG. 3 is a perspective view of a fastener 100 embodying the present invention. The fastener 100 generally includes a first member 102, a second member 104, and a connecting member 106. As may be noted in FIG. 3, the first member 102 and second member 104 are substantially parallel to each other and substantially perpendicular to the connecting member 106 which connects the first member 102 to the second member 104.

The first member 102 is generally cylindrical or can have any other shape. It has a longitudinal axis 108 and a through channel 112 along the longitudinal axis 108.

The first member 102 also includes a first end 116 and a second end 118. Similarly, the second member 104 includes a first end 120 and a second end 122. The first end 116 of member 102 forms a pointed dilation tip 124. The dilation tip 124 may be conical and more particularly takes the shape of a truncated cone. The tip can also be shaped to have a cutting edge in order to reduce tissue resistance.

The first and second members 102 and 104 and the connecting member 106 may be formed of different materials and have different textures. These materials may include, for example, plastic materials such as polypropylene, polyethylene, polyglycolic acid, polyurethane, or a thermoplastic elastomer. The plastic materials may include a pigment contrasting with body tissue color to enable better visualization of the fastener during its deployment. Alternatively, the fastener may be formed of a malleable metal with shape memory, such as Nitinol.

As may be further noted in FIG. 3, the connecting member 106 has a vertical dimension 128 and a horizontal dimension 130 which is transverse to the vertical dimension. The horizontal dimension is substantially less than the vertical dimension to render the connecting member 106 readily bendable in a horizontal plane. The connecting member is further rendered bendable by the nature of the material from which the fastener 100 is formed. The connecting member may be formed from either an elastic plastic or a permanently deformable plastic. An elastic material would prevent compression necrosis in some applications.

It may be noted in FIG. 3 that the first member 102 has a continuous lengthwise slit 125 extending between the first and second ends 116 and 118. The slit 125 is continuous from the first end 116 to the second end 118. The slit 125 has a transverse dimension which, as will be seen subsequently, along with the flexibility of the member 102, permits the fastener 100 to be released from the stylet. More specifically, because the fastener number 102 is formed of flexible material, the slit 125 may be made larger through separation to allow the deployment stylet to be released from the fastener 100 through the slit 125 as will be seen subsequently. The slit 125 also permits the fastener to be snap mounted on the stylet before deployment. The slit 125 extends substantially parallel to the through channel 112 and the center axis 108 of the first member 102. It may also be noted that the slit 125 has a width dimension that is smaller or less than the diameter of the through channel 112. This assures that the fastener 100 will remain on the tissue piercing deployment stylet as it is pushed towards and into the tissue as will be seen subsequently.

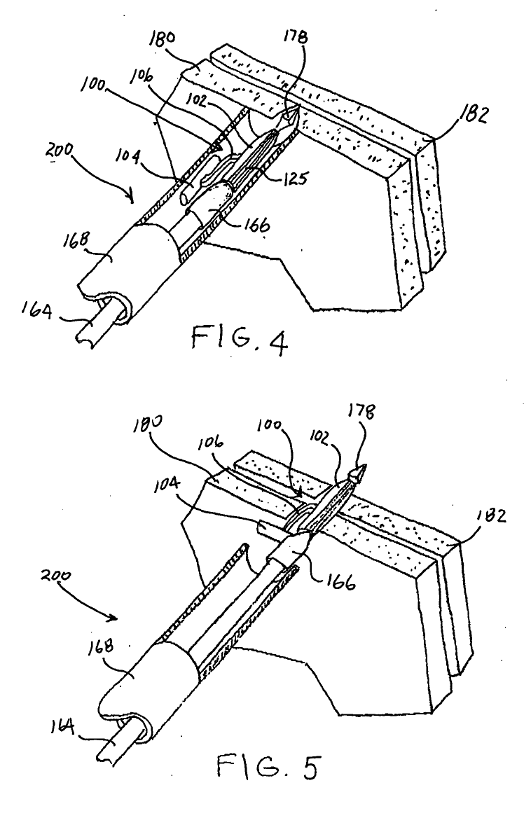

Referring now to FIG. 4, it is a perspective view with portions cut away of a fastener assembly 200 embodying the present invention for deploying the fastener 100. The tissue layer portions above the fastener 100 have been shown cut away in FIGS. 4-8 to enable the deployment procedure to be seen more clearly. The assembly 200 generally includes the fastener 100, a deployment stylet 164, and a guide tube 168.

The first member 102 of the fastener 100 is slidingly received on the end of the deployment stylet 164. The deployment stylet 164 has a pointed tip 178 for piercing the tissue layers 180 and 182 to be fastened together. The stylet 164 has an enlarged engagement structure 166 proximal to the tip 178 having at least a portion with a cross-sectional dimension greater than that of the through channel for making an interference fit with through channel 112. This permits the stylet 164 to engage the member 102 and push the fastener member 102 through the tissue layers 180 and 182. It also serves to later separate or enlarge the slit 125 to release the stylet from the member 102 at the end of the deployment. The tissue piercing stylet 164, and the fastener 100 are both within the guide tube 168. The guide tube 168 may take the form of a catheter, for example, as previously mentioned, or a guide channel within a block of material.

As will be further noted in FIG. 4, the second member 104 is disposed along side the first member 102. This is rendered possible by the flexibility of the connecting member 106.

With the first member 102 of the fastener 100 received on the tissue piercing stylet 164 and with the engagement structure 166 engaging the first member 102, the stylet may be translated in a distal direction towards the tissue to cause the tip 178 of the tissue piercing stylet 164 to pierce the tissue layers 180 and 182. The tissue piercing stylet 164 and fastener 100 are guided to the tissue layers 180 and 182 by the guide tube 168.

As shown in FIG. 6, the tip 178 of the tissue piercing stylet 164 has pierced the tissue layers 180 and 182 and continued advancement of the stylet 164 has pushed the first member 102 of the fastener 100 through the tissue layers 180 and 182. This may be accomplished during a smooth single continuous stroke of the stylet 164. As may be further seen in FIG. 6, continued forward movement of the stylet 164 has caused member 102 to pass entirely through tissue layers 180 and 182. The engagement structure 166 has also pierced the tissue and the second member 104 has engaged the tissue layer 180.

As will be still further noted in FIG. 6, the engagement structure 166 has a conical surface 167 and thus, the engagement structure 166 increases in dimension in the proximal direction. The conical surface 167 permits the engagement portion 166 to gradually enlarge the slit 125.

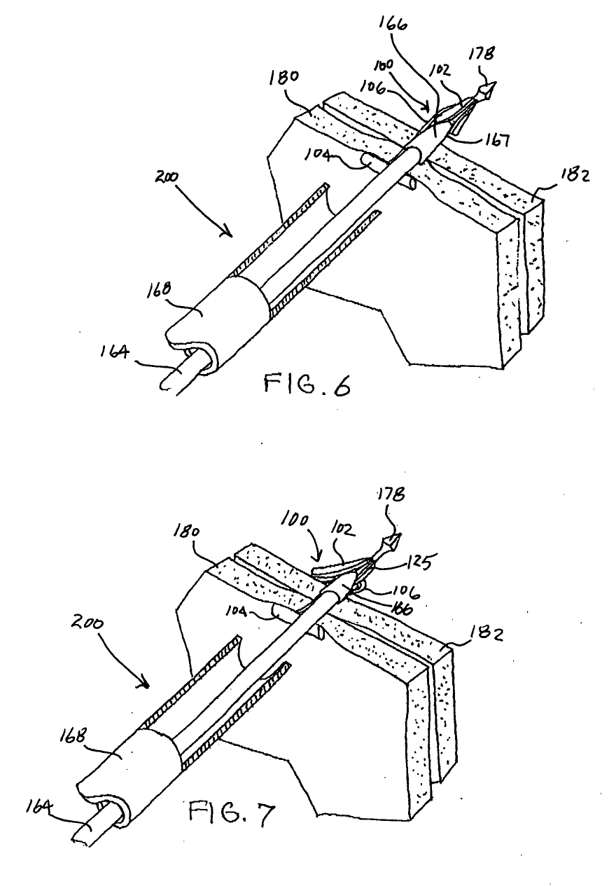

Eventually, with further continued forward movement of stylet 164, the slit 125 becomes wide enough to permit the stylet 164 to be released from the member 102 and more particularly, the through channel 112, through the enlarged slit 125. FIG. 7 shows the assembly 200 with the stylet 164 just about totally released from the member 102. Engagement of the second member 104 with the tissue 180 assists in this process by holding the fastener 100 from substantially forward movement.

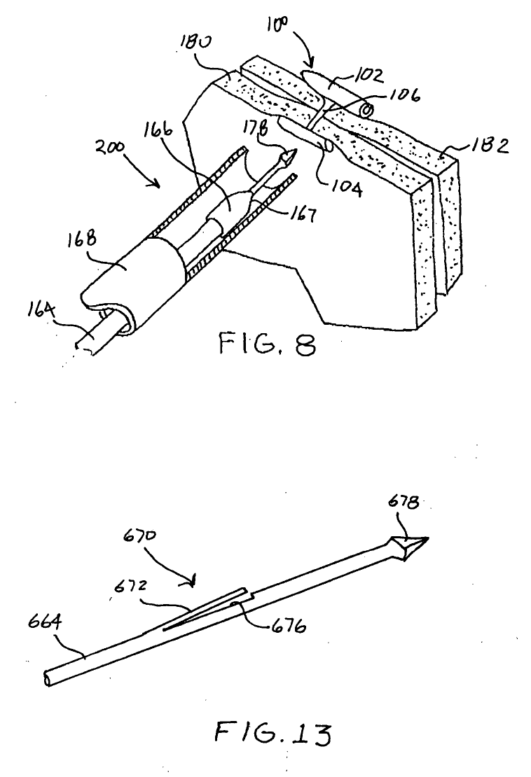

FIG. 8 illustrates the fastener 100 in its fully deployed position. It will be noted that the fastener has returned to its original shape. The tissue layers 180 and 182 are fastened together between the first member 102 of the fastener 100 and the second member 104 of the fastener 100. The connecting member 106 extends through the tissue layers 180 and 182.

The deployment and release of the fastener 100 from the stylet 164 is made possible with but a single forward stroke of the stylet. This minimizes the number of elements which must be manipulated or controlled during deployment of the fastener 100.

FIGS. 9 and 10 show further fasteners 300 and 400 which may be employed in accordance with further embodiments of the invention. In these side views, only the first members 302 and 402 respectively are shown as it is contemplated that each fastener would include a second member and connecting member similar or identical to the second member 104 and connecting member 106 of FIG. 3.

In FIG. 9, the second member 302 includes a web 308 of material bridging across the slit 325. The web 308 may be breakable by the stylet and thus provide a resistance against enlargement of the slit 325 after fastener deployment and as the stylet is pushed forward to be released from the member 302. The thickness of the web 308 may be selected to require a preselected controlled force necessary for breaking the web to cause fastener release.

FIG. 10 shows a fastener 400 wherein its first member 402 has a slit 425 that continuously increases in width along the fastener in the distal direction. This increase in slit dimension may be helpful to reduce the force required for fastener release.

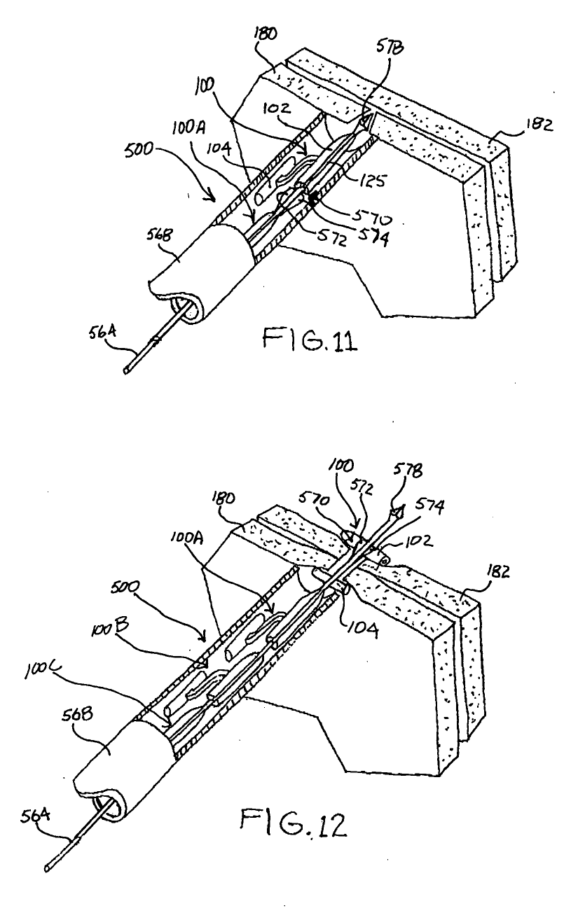

FIGS. 11 and 12 are perspective views with portions cut away of another fastener assembly 500 according to a further embodiment of the present invention deploying the fastener 100. The tissue layer portions above the fastener 100 have been shown cut away in FIGS. 11 and 12 to enable the deployment procedure to be seen more clearly. The assembly 500 generally includes the fastener 100, a deployment stylet 564, and a guide tube 568. The assembly 500 includes additional fasteners 100A, shown in FIGS. 11 and 12, and fasteners 100B and 100C, visible in FIG. 12, which are slidingly received on the stylet 564.

The first member 102 of the fastener 100 is slidingly received on the deployment stylet 564. The pointed tip 578 of the stylet 564 is piercing the tissue layers 180 and 182. The stylet 564 has an engagement structure proximal to the tip 578 taking the form and function of a spring loaded latch 570 having spring loaded wings 572 and 574. When forced outwardly by a spring (not shown) internal to the stylet 564, the wings 572 and 574 present a cross-sectional dimension greater than that of the through channel 112 of the fastener 100 (FIG. 3) for making an interference fit therewith. This permits the stylet 564 to engage the member 102 and push the fastener member 102 through the tissue layers 180 and 182. It also serves to later separate or enlarge the slit 125 as previously described to release the stylet from the member 102 at the end of the deployment. The tissue piercing stylet 564, and the fastener 100 are guided by the guide tube 568. The guide tube 568 may, as previously mentioned, take the form of a catheter, for example, or a guide channel within a block of material. As may further be noted in FIG. 11, the second member 104 is again disposed along side the first member 102.

As previously described with respect to the embodiment of FIGS. 4-8, with the latch 570 engaging the first member 102, the stylet may be translated in a distal direction towards the tissue to cause the tip 578 to pierce the tissue layers 180 and 182. Continued advancement of the stylet 564 will push the first member 102 of the fastener 100 through the tissue layers 180 and 182 in a smooth single continuous stroke of the stylet 564. Continued forward movement of the stylet 564 will cause member 102 to pass entirely through tissue layers 180 and 182, the second member 104 to engage the tissue layer 180, and the latch 570 to gradually enlarge the slit 125 until the slit 125 becomes wide enough to permit the stylet 564 to be released from the member 102. FIG. 12 shows the assembly 500 with the stylet 564 totally released from the member 102. Once again, engagement of the second member 104 with the tissue 180 assists in this process by holding the fastener as the latch 570 opens the slit 125 for release of the stylet 564.

It will be noted that the fastener 100 has returned to its original shape. The tissue layers 180 and 182 are fastened together between the first and second members 102 and 104 of the fastener 100.

The deployment and release of the fastener 100 from the stylet 164 is made possible with but a single forward stroke of the stylet. The next fastener 100A may now be advanced over the spring loaded latch into a loaded position for engagement by the latch. When the fastener 100A passes over the latch 570, the wings 572 and 574 will retract into the body of the stylet against the spring force. When the fastener reaches its loaded position, the wings 572 and 574 snap out to engage the fastener. Fastener 100A is then ready to be deployed.,

As will be appreciated by those skilled in the art, the wings 572 and 574 of the spring loaded latch may be alternatively formed of resilient wire. The proximal ends of the wires would be welded to the stylet. The wire may be configured to take an unstressed shape corresponding to the shape of the wings 572 and 574. This would negate the need for providing a spring or springs within the body of the stylet.

A further embodiment of a stylet having a spring loaded latch is shown in FIG. 13. The stylet 664 of FIG. 13 includes a pointed tip 678 as previously described and an integral spring loaded latch 670. The latch 670 includes a spring arm 672 which permits fasteners to slide thereover for loading. When the fasteners slide over the latch, the spring arm 672 is forced into a notch 676 resulting from the formation of spring arm 672. With spring arm 672 is within the notch 676, the fasteners are free to slide distal to the latch 670. When the fasteners clear the latch, the spring arm 672 springs back to the illustrated configuration. It is now ready to engage the loaded fastener, drive it into the tissue, and separate the fastener from the stylet in a single stroke of the stylet in a manner as previously described.

While the invention has been described by means of specific embodiments and applications thereof, it is understood that numerous modifications and variations may be made thereto by those skilled in the art without departing from the spirit and scope of the invention. It is therefore to be understood that within the scope of the claims, the invention may be practiced otherwise than as specifically described herein.

What is claimed is:

1. A fastener assembly for use in a mammalian body, comprising:
a plurality of fasteners, each fastener being arranged to fasten tissue when deployed; and
a stylet that guides each fastener into tissue to be fastened, the plurality of fasteners being carried on the stylet prior to deployment.

2. The assembly of claim 1, wherein each of the fasteners includes a through channel that slidingly receives the stylet.

3. The assembly of claim 1, wherein each of the fasteners includes a first member, a second member, and a connecting member connecting the first member to the second member, the first and second members each having first and second ends, and wherein the connecting member connects to the first and second members intermediate the first and second ends of each of the first and second members.

4. The assembly of claim 1, further comprising a drive structure that drives the fasteners into the tissue while the fasteners are carried on the stylet.

5. The assembly of claim 1, wherein the stylet includes a pointed tip that pierces the tissue before the fasteners pierce the tissue.

6. The assembly of claim 1, wherein the fasteners include a driven member that is driven into the tissue while on the stylet during deployment, a second member, and a connecting member connecting the driven member and second member, and wherein the tissue is between the driven member and the second member and the connecting member extends through the tissue when the fasteners are deployed.

7. The assembly of claim 6, wherein the driven member of each fastener is releasable from the stylet.

8. The assembly of claim 7, wherein the driven member of the fasteners includes a through channel that slidingly receives the stylet and that permits the fasteners to be carried on the stylet.

9. The assembly of claim 8, wherein the fasteners include a slit communicating with their through channel and wherein the fasteners are releasable from the stylet by the stylet passing through their slit.

10. The assembly of claim 1, further comprising a guide defining a guide lumen adapted to receive the fasteners and stylet and that guides the stylet and fasteners to the tissue.

11. A fastener assembly for use in a mammalian body, comprising:
a plurality of fasteners, each fastener being arranged to fasten tissue when deployed and including a driven member, a second member, and a connecting member connecting the driven member to the second member, the driven and second members each having first and second ends with the connecting member connecting to the driven and second members intermediate the first and second ends of each of the driven and second members, the driven member including a through channel; and
a stylet that guides each fastener into tissue to be fastened, the plurality of fasteners being carried on the stylet with the through channel of the driven member of each fastener slidingly received on the stylet.

12. The assembly of claim 11, wherein the stylet includes a drive structure that drives each fastener into the tissue.

13. The assembly of claim 12, wherein the stylet includes a pointed tip that pierces the tissue before the fasteners pierce the tissue.

14. The assembly of claim 11, wherein the driven member of each fastener is releasable from the stylet.

15. The assembly of claim 11, wherein each drive member of each fastener includes a slit communicating with its through channel and wherein the fasteners are releasable from the stylet by the stylet passing through the slits.

16. The assembly of claim 11, further comprising a guide defining a guide lumen adapted to receive the fasteners and stylet and that guides the stylet and fasteners to the tissue.
